(19) United States
(12) Patent Application Publication
(10) Pub. No.: US 2021/0244493 A1
(43) Pub. Date: Aug. 12, 2021

(54) ELECTROSURGICAL SYSTEM, ELECTROSURGICAL CONTROL UNIT, ELECTROSURGICAL INSTRUMENT, AND METHOD OF OPERATING AN ELECTROSURGICAL SYSTEM

(71) Applicant: OLYMPUS WINTER & IBE GMBH, Hamburg (DE)

(72) Inventors: Anne KWIK, Berlin (DE); Dirk Bombor, Königs Wusterhausen (DE)

(73) Assignee: OLYMPUS WINTER & IBE GMBH, Hamburg (DE)

(21) Appl. No.: 17/173,038

(22) Filed: Feb. 10, 2021

(30) Foreign Application Priority Data
Feb. 10, 2020 (DE) 10 2020 103 307.9

(51) Int. Cl. A61B 90/00; A61B 18/12; A61B 18/14; A61B 90/98; G16H 20/40; G16H 40/63; H04L 9/08; H04L 9/32

(52) U.S. Cl. CPC A61B 90/08; A61B 18/1206; A61B 18/14; A61B 90/98; A61B 2017/00225; G16H 40/63; H04L 9/0894; H04L 9/32; A61B 2090/0805; G16H 20/40

(57) ABSTRACT

An electrosurgical system is presented, comprising: an electrosurgical control unit (10), at least one electrosurgical instrument (11, 12) connectable to the electrosurgical control unit (10), wherein the at least one electrosurgical instrument (11, 12) comprises a first memory element (30, 31) on which data characterizing the electrosurgical instrument (11, 12) is or can be stored, the at least one electrosurgical instrument (11, 12) is configured after being connected to the electrosurgical control unit (10), to transmit the data characterizing the electrosurgical instrument (11, 12) at least in part as first data to the electrosurgical control unit (10), and the electrosurgical control unit (10) is configured to determine allowable operating parameters of the electrosurgical instrument (11, 12) on the basis of the first data transmitted by the electrosurgical instrument (11, 12).

The electrosurgical system is characterized in that data authenticating the electrosurgical instrument (11, 12) is stored or can be stored on the first memory element (30, 31) or a second memory element (40, 41) of the electrosurgical instrument (11, 12), the electrosurgical control unit (10) is configured to transmit second data to the electrosurgical instrument (11, 12) after being connected to the electrosurgical instrument (11, 12), the at least one electrosurgical instrument (11, 12) is configured to transmit third data derived from the second data transmitted by the electrosurgical control unit (10) and the data authenticating the electrosurgical instrument (11, 12) to the electrosurgical control unit (10), and the electrosurgical control unit (10) is configured to determine an allowable range of functions for the electrosurgical instrument (11, 12) on the basis of the third data transmitted by the electrosurgical instrument (11, 12).

The invention relates to an electrosurgical system comprising an electrosurgical control unit and at least one electrosurgical instrument which is connectable to the electrosurgical control unit, wherein: the at least one electrosurgical instrument comprises a first memory element on which data characterizing the electrosurgical instrument is or can be stored; the at least one electrosurgical instrument is configured to transmit, after connection to the electrosurgical control unit, the data characterizing the electrosurgical instrument at least partially as first data to the electrosurgical control unit; and the electrosurgical control unit is configured to determine allowable operating parameters of the electrosurgical instrument based on the first data transmitted by the electrosurgical instrument.

The invention further relates to an electrosurgical control unit, an electrosurgical instrument, and a method of operating an electrosurgical system.

Electrosurgical systems are used in medicine for treating tissue of a human or animal patient. In most cases, a high-frequency alternating current is generated in the electrosurgical control unit and is directed to the electrosurgical instrument, where it is introduced into the tissue by one or more treatment electrodes. In other methods, the alternating current generates a plasma in a fluid, which is then brought into contact with the tissue.

In some electrosurgical systems, the alternating current is used to excite ultrasonic vibration in the electrosurgical instrument, which is then introduced into the tissue to be treated via a sonotrode.

Occasionally, direct current is also used in electrosurgical systems.

The electrosurgical control unit can be, for example, an electrosurgical generator or an ultrasound generator.

In the course of technical development, numerous novel electrosurgical instruments have been developed which are particularly suitable for certain surgical procedures. For such novel instruments, special waveforms for the high-frequency alternating current have often been defined in order to be able to use them particularly efficaciously.

In order to be able to control as many different electrosurgical instruments as possible, modern electrosurgical control units are therefore able to provide a correspondingly high number of waveforms at different electrical powers.

However, not every waveform may be compatible with every instrument. For safe operation of an electrosurgical system, it is therefore necessary for the control unit to provide only those waveforms and powers that the connected instrument can process.

For this purpose, it is known to equip the electrosurgical instrument with a memory on which data characterizing the electrosurgical instrument are stored. This data may be transmitted to the control unit when the instrument is connected to the control unit, and the control unit may use this data to define allowable operating parameters of the electrosurgical instrument.

The data characterizing the electrosurgical instrument may be a simple identification code, such as a type designation. The characterizing data may also directly include allowable operating parameters, such as allowable voltages and/or powers of an AC current to be delivered by the control unit.

Electrosurgical systems implemented according to the prior art can provide reliable and safe operation if it is ensured that the data stored on the memory of the electrosurgical instrument accurately characterizes the corresponding electrosurgical instrument. This is verified by electrosurgical instrument manufacturers before the instrument is placed on the market.

However, problems may arise when an electrosurgical instrument does not originate from a trusted manufacturer. For example, it is a common situation for novel electrosurgical instruments to be replicated by third-party manufacturers and marketed as counterfeit products. Such counterfeit instruments may be provided with characterizing data specifying operating data for which the instrument may not be suitable due to insufficient manufacturing quality. The use of such a counterfeit electrosurgical instrument in an electrosurgical system may lead to inadequate treatment results or even to hazards for a patient or an attending physician.

It is therefore an object of the invention to provide an electrosurgical system which is improved with respect to the described problem.

This object is achieved according to a first aspect of the invention by an electrosurgical system comprising an electrosurgical control unit and at least one electrosurgical instrument which is connectable to the electrosurgical control unit, wherein: the at least one electrosurgical instrument comprises a first memory element on which data characterizing the electrosurgical instrument is or can be stored; the at least one electrosurgical instrument is configured to transmit, after being connected to the electrosurgical control unit, the data characterizing the electrosurgical instrument at least partially as first data to the electrosurgical control unit and the electrosurgical control unit is configured to determine allowable operating parameters of the electrosurgical instrument on the basis of the first data transmitted from the electrosurgical instrument; wherein data authenticating the electrosurgical instrument is or can be stored on the first memory element or a second memory element of the electrosurgical instrument; the electrosurgical control unit is configured to transmit second data to the electrosurgical instrument after being connected to the electrosurgical instrument; the at least one electrosurgical instrument is configured to transmit third data derived from the second data which are transmitted by the electrosurgical control unit and from the data which authenticate the electrosurgical instrument to the electrosurgical control unit; and the electrosurgical control unit is configured to set an allowable range of functions for the electrosurgical instrument on the basis of the third data which are transmitted by the electrosurgical instrument.

By this, the electrosurgical control unit can identify whether the electrosurgical instrument originates from a trusted manufacturer. For this purpose, the data authenticating the electrosurgical instrument is not transmitted directly to the electrosurgical control unit, so that this data cannot be easily copied and used for counterfeit electrosurgical instruments.

If the electrosurgical control unit detects, based on the third data, that the instrument is an authentic electrosurgical instrument, the instrument may be operated using the operating parameters determined based on the first data. On the other hand, if the instrument is not an authentic electrosurgical instrument, operation may be limited to some simple functions. Similarly, the electrosurgical control unit may be set up to completely reject a non-authentic electrosurgical instrument, i.e., not allow any functions at all.

In a further embodiment of an electrosurgical system according to the invention, the at least one electrosurgical instrument may be configured to determine the third data according to a cryptographic method from the second data and the data authenticating the electrosurgical instrument.

In the context of the invention, a cryptographic method is understood to be a method which cannot be reversed or can be reversed only with great effort. It is thus made more difficult to determine the authenticating data even if the second data and the third data are known, for example by eavesdropping on the communication between the electrosurgical control unit and the electrosurgical instrument.

The electrosurgical control unit may be configured to determine the second data, at least in part, in accordance with a random process. This makes it more difficult to generate a list of second data and associated allowable third data by eavesdropping on communications between the electrosurgical control unit and the electrosurgical instrument.

In one possible embodiment of an electrosurgical system according to the invention, a cryptographic key may be stored in the first or second memory element. The cryptographic key may be part of the data authenticating the electrosurgical instrument.

In one embodiment of an electrosurgical system according to the invention, the electrosurgical control unit may include a third memory element on which a copy of the cryptographic key is stored.

In another embodiment of an electrosurgical system according to the invention, the cryptographic key may be part of a cryptographic key pair, and the electrosurgical control unit may comprise a third memory element on which the other part of the cryptographic key pair is stored.

Accordingly, the cryptographic method may be a symmetric method or an asymmetric method.

In an embodiment of an electrosurgical system according to the invention, the system may comprise a plurality of electrosurgical instruments, each of which comprises a first memory element having a cryptographic key stored therein, and in the third memory element a copy of each of the cryptographic keys or, for each cryptographic key, the other portion of the corresponding cryptographic key pair may be stored.

In this way, an electrosurgical control unit may be operated with different electrosurgical instruments.

According to a second aspect of the invention, the object is achieved by an electrosurgical control unit of an electrosurgical system according to the above embodiments.

According to a third aspect of the invention, the object is achieved by an electrosurgical instrument of an electrosurgical system according to the above embodiments.

With respect to the advantages and effects obtainable thereby, explicit reference is made in each case to what has been said above.

The object is achieved according to a fourth aspect of the invention by a method for operating an electrosurgical system comprising an electrosurgical control unit and at least one electrosurgical instrument, comprising the steps: Connecting the electrosurgical control unit to the at least one electrosurgical instrument; transmitting first data characterizing the electrosurgical instrument to the electrosurgical control unit; and determining allowable operating parameters for the electrosurgical instrument based on the first data; transmitting second data from the electrosurgical control unit to the electrosurgical instrument; deriving third data from the second data and data authenticating the electrosurgical instrument stored on the electrosurgical instrument; transmitting the third data to the electrosurgical control unit; and determining, by the electrosurgical control unit, an allowable range of functions for the electrosurgical instrument based on the third data.

The third data may be derived from the second data and the data authenticating the electrosurgical instrument according to a cryptographic method.

The second data may be determined according to a random process.

The invention is explained in more detail below with reference to an exemplary FIGURE. In this regard, the exemplary embodiments illustrated below are merely intended to contribute to a better understanding of the invention without limiting it.

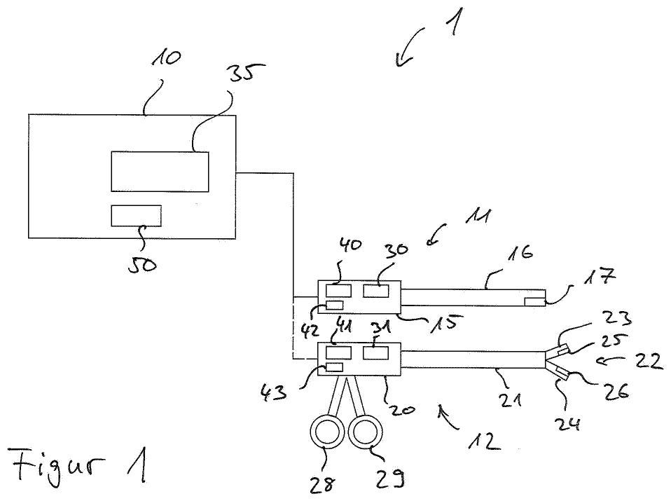

FIG. 1 shows an electrosurgical system.

FIG. 1 shows an electrosurgical system 1. The electrosurgical system 1 comprises an electrosurgical control unit 10, which in the example shown is an electrosurgical generator. Further, the electrosurgical system 1 comprises two electrosurgical instruments 11, 12 that may be alternately or simultaneously connected to the electrosurgical control unit 10.

The electrosurgical instrument 11 includes a main body 15, which can serve as a handle, and an elongated shaft 16, at the distal end of which an electrode 17 is disposed. The electrosurgical instrument 11 may be, for example, a monopolar electrosurgical scalpel.

The electrosurgical instrument 12 also includes a main body 20 and an elongated shaft 21. Unlike the electrosurgical instrument 11, the electrosurgical instrument 12 includes a forceps jaw 22 with movable branches 23, 24. The branches 23, 24 each include a treatment electrode 25, 26. Further arranged on the main body 20 are two handle levers 28, 29 which are movable relative to each other for actuating the branches 23, 24. The electrosurgical instrument 12 may be, for example, a bipolar coagulation forceps.

The electrosurgical instruments 11, 12 are each equipped with a first memory element 30, 31 which can be read out by the electrosurgical control unit 10 after the electrosurgical instruments 11, 12 are connected to the electrosurgical control unit 10. For this purpose, the electrosurgical control unit 10 comprises a controller 35. Data characterizing the electrosurgical instruments 11, 12 are stored on the first memory elements 30, 31. These data may include, for example, type designations of the electrosurgical instruments 11, 12, and/or directly allowable parameters of an alternating current to be delivered to the electrosurgical instruments 11, 12 by the electrosurgical control unit 10.

Based on the data read from the first memory elements 30, 31, the controller 35 determines the operating parameters with which the electrosurgical instruments 11, 12 may be operated. The electrosurgical control unit 10 may include a user interface, not shown, through which these operating parameters may be further adjusted by a user of the electrosurgical system 1. For example, based on the data read from the first memory elements 30, 31, the controller 35 may define a framework within which the operating parameters may be freely selected by a user via the user interface.

In order to ensure that the electrosurgical instruments 11, 12 originate from a trustworthy source, they additionally comprise second memory elements 40, 41 on which data is stored that can be used to authenticate the electrosurgical instruments 11, 12. The authentication data stored on the memory elements 40, 41 may be cryptographic keys.

In order to avoid direct transmission of the authentication data, which could in principle be eavesdropped, a controller 42, 43 is associated with each of the second memory elements 40, 41. The controllers 42, 43 are set up to receive request data, to link these via a cryptographic function with authentication data stored on the memory element 40 and 41 respectively, and to output the result of the link as response data.

Two possible authentication methods for the electrosurgical instruments 11, 12 are shown below.

A first authentication method is based on a symmetric encryption method. In this method, a cryptographic key K1 is stored on the second memory element 40 of the electrosurgical instrument 11, and a copy of the key K1 is stored in a third memory element 50 of the electrosurgical control unit 10.

Now, when the electrosurgical instrument 11 is connected to the electrosurgical control unit 10, the controller 35 first generates a random value Z and sends it to the electrosurgical instrument 11. In the electrosurgical instrument 11, the controller 42 receives the random value Z and encrypts it with the key K1 stored on the second memory element 40 to form a return value R=f(Z,K1). The return value R is transmitted back to the controller 35.

The controller 35 now reads the copy of the key K1 from the third memory element 50 and uses it to decrypt the return value R according to the inverse function f⁻¹. If the result of the decryption Z′=f⁻¹(R,K1) corresponds to the original random value Z, the electrosurgical instrument 11 is successfully authenticated. Otherwise, the electrosurgical instrument 11 may be a counterfeit. In the latter case, the controller 35 may restrict a possible range of functions for the electrosurgical instrument 11 in order to avoid endangering the patient or the user of the electrosurgical system 1. The control 35 may also completely prevent operation of the electrosurgical instrument 11.

A second key K2 may be stored in the second memory element 41 of the electrosurgical instrument 12, and a copy of the second key K2 is also stored in the third memory element 50. Thus, the electrosurgical control unit 10 can authenticate multiple types of electrosurgical instruments 11, 12.

Available symmetric encryption methods are, for example, “AES”, “Blowfish” or “Twofish”.

A second possible authentication method is based on an asymmetric encryption method. Herein, a private key P1 is stored on the second memory element 40 of the electrosurgical instrument 11, whereas a corresponding public key O1 is stored on the third memory element 50.

In order to authenticate the electrosurgical instrument 11, the controller 35 first generates a random value Z again, encrypts it according to the function A=g(Z,O1) to obtain the request value A, and sends the request value A to the electrosurgical instrument 11.

The controller 42 receives the request value A and decrypts it using the private key P1 according to the function R=g′(A,P1) to obtain the return value R, and sends the return value R back to the electrosurgical control unit 10.

The controller 35 now checks whether the return value R corresponds to the random value Z. If this is the case, the electrosurgical instrument 11 has been successfully authenticated, otherwise a partial or complete restriction of the provided range of functions is effected as described above.

For multiple types of electrosurgical instruments 11, 12, multiple key pairs O1, P1, O2, P2 can be used similar to the embodiments above.

The utilization of an asymmetric encryption method has the advantage that when newly developed electrosurgical instruments are introduced for which a new cryptographic key is to be used, the associated public key can be distributed in a straightforward manner to electrosurgical control units located in the field without the need to reveal the private key for this purpose. If a symmetric encryption method is used, it must be ensured that the key does not fall into the wrong hands when distributing the new key to electrosurgical control units in the field.

Possible asymmetric encryption methods are for example “RSA” or the “Elgamal” method.

In order to prevent access to the memory contents of the second memory elements 40, 41, these are combined with the associated controllers 42 or 43 to form a security module. This can be, for example, a “Trusted Platform Module” (TPM) according to the specification of the “Trusted Computing Group” (TCG).

To simplify the design of the electrosurgical instruments 11, 12, the first and second memory elements 30 and 40 or 31 and 41 can be combined into a single memory element.

1. An electrosurgical system comprising
an electrosurgical control unit; and
at least one electrosurgical instrument connectable to the electrosurgical control unit, wherein:
the at least one electrosurgical instrument comprises a first memory element on which data characterizing the electrosurgical instrument is or can be stored;
the at least one electrosurgical instrument is configured to transmit, after being connected to the electrosurgical control unit, the data characterizing the electrosurgical instrument at least partially as first data to the electrosurgical control unit; and
the electrosurgical control unit is configured to determine allowable operating parameters of the electrosurgical instrument based on the first data transmitted by the electrosurgical instrument;
wherein
data authenticating the electrosurgical instrument is or can be stored on the first memory element or on a second memory element of the electrosurgical instrument,
the electrosurgical control unit is configured to transmit second data to the electrosurgical instrument after being connected to the electrosurgical instrument,
the at least one electrosurgical instrument is configured to transmit third data which is derived from the second data transmitted by the electrosurgical control unit and the data authenticating the electrosurgical instrument to the electrosurgical control unit, and
the electrosurgical control unit is configured to determine an allowable range of functions for the electrosurgical instrument based on the third data transmitted from the electrosurgical instrument.

2. Electrosurgical system according to claim 1, wherein the at least one electrosurgical instrument is arranged to determine the third data according to a cryptographic method from the second data and the data authenticating the electrosurgical instrument.

3. Electrosurgical system according to claim 1, wherein the electrosurgical control unit is arranged to determine the second data at least partially according to a random process.

4. Electrosurgical system according to claim 2, wherein a cryptographic key is stored on the first and/or second memory element.

5. Electrosurgical system according to claim 4, wherein the electrosurgical control unit comprises a third memory element on which a copy of the cryptographic key is stored.

6. Electrosurgical instrument according to claim 4, wherein the cryptographic key is part of a cryptographic key pair, and that the electrosurgical control unit comprises a third memory element on which the other part of the cryptographic key pair is stored.

7. Electrosurgical system according to claim 5, wherein the system comprises a plurality of electrosurgical instruments each comprising a first or second memory element with a cryptographic key stored thereon, and that in the third memory element a copy of each of the cryptographic keys or, for each cryptographic key, the other part of the corresponding cryptographic key pair is stored.

8. Electrosurgical control unit of an electrosurgical system according to claim 1.

9. Electrosurgical instrument of an electrosurgical system according to claim 1.

10. Method for operating an electrosurgical system comprising an electrosurgical control unit and at least one electrosurgical instrument, comprising the steps of:
connecting the electrosurgical control unit to the at least one electrosurgical instrument,
transmitting first data characterizing the electrosurgical instrument to the electrosurgical control unit, and
determining allowable operating parameters for the electrosurgical instrument based on the first data,
transmitting second data from the electrosurgical control unit to the electrosurgical instrument,
deriving third data from the second data and data stored on the electrosurgical instrument authenticating the electrosurgical instrument,
transmitting the third data to the electrosurgical control unit, and
determining, by the electrosurgical control unit, an allowable range of functions for the electrosurgical instrument based on the third data.

11. Method according to claim 10, wherein the third data is derived according to a cryptographic method from the second data and the data authenticating the electrosurgical instrument.

12. Method according to claim 11, wherein the second data is determined according to a random method.
